(19) United States
(12) Patent Application Publication
(10) Pub. No.: US 2023/0302078 A1
(43) Pub. Date: Sep. 28, 2023

(54) METHODS AND COMPOSITIONS FOR WEIGHT CONTROL

(71) Applicant: 4Life Patents, LLC, Sandy, UT (US)

(72) Inventors: Brent Vaughan, Draper, UT (US); David Vollmer, South Jordan, UT (US); Paula Brock, Salt Lake City, UT (US); Shane Lefler, American Fork, UT (US)

(21) Appl. No.: 18/197,700

(22) Filed: May 15, 2023

Related U.S. Application Data
(62) Division of application No. 15/464,296, filed on Mar. 20, 2017, now abandoned.
(60) Provisional application No. 62/406,935, filed on Oct. 11, 2016.
(60) Provisional application No. 62/310,561, filed on Mar. 18, 2016.

(51) Int. Cl. A61K 36/752; A61K 36/81; A61K 36/185; A61K 36/53; A61K 31/216; A61K 38/01; G01N 33/483; A23L 33/18; A23L 33/00; A23L 33/105; A23L 33/17

(52) U.S. Cl. CPC A61K 36/752; A61K 36/81; A61K 36/185; A61K 36/53; A61K 31/216; A61K 38/012; G01N 33/4833; A23L 33/18; A23L 33/40; A23L 33/105; A23L 33/30; A23L 33/17; A23V 2002/00

(57) ABSTRACT

Methods and compositions for providing control over a subject's body, include methods and compositions for enhancing the ability of a subject's body to lose weight, or for inducing weight loss in the subject's body. Such methods and compositions may induce thermogenesis in the adipocytes of a subject's body, enhancing the subject's metabolism, inhibit adipogenesis in adipocytes of the individual, and reduce the subject's cravings for food, the subject's appetite and/or the amount of food consumed by the subject. Such a composition may include African mango (Irvinia gabonensis) seed extract, citrus fruits extract from Citrus aurantium, Citrus sinensis, and/or Citrus paradisi (standardized to 5% synephrine and 80% bioflavonoids), Coleus forskholi root extract, and a source of dihydrocapsiate. The composition may be administered with a protein supplement, such as a whey protein supplement (e.g., a hydrolyzed whey protein supplement).

### CROSS-REFERENCE TO RELATED APPLICATIONS

This application is a divisional of U.S. patent application Ser. No. 15/464,296, filed on May 20, 2017 and titled METHODS AND COMPOSITIONS FOR WEIGHT CONTROL (“the '296 Application”). The '296 Application includes claims to the benefit of the Mar. 18, 2016 filing date of U.S. Provisional Patent Application 62/310,561, titled METHODS AND COMPOSITIONS FOR WEIGHT CONTROL (“the '561 Provisional Application”) and the Oct. 11, 2016 filing date of U.S. Provisional Patent Application No. 62/406,935, titled METHODS AND COMPOSITIONS FOR WEIGHT CONTROL (“the '935 Provisional Application”), which were made pursuant to 35 U.S.C. § 119(e). The entire disclosures of the '296 Application, the '561 Provisional Application, and the '935 Provisional Application are hereby incorporated herein.

### TECHNICAL FIELD

This disclosure relates generally to methods and compositions for providing control over a subject's body, including, but not limited to, methods and compositions for enhancing the ability of a subject's body to lose weight, or for inducing weight loss in the subject's body. More specifically, this disclosure relates to methods and compositions for inducing thermogenesis in the adipocytes of a subject's body, enhancing the subject's metabolism, inhibiting adipogenesis in adipocytes of the individual, and reducing the subject's cravings for food, the subject's appetite and/or the amount of food consumed by the subject.

### SUMMARY

Methods for enhancing the ability of a subject, such as an individual, to lose weight are disclosed. Such a method may include administering or otherwise providing inventive combinations of naturally occurring substances, including nutritional supplements, to the subject. The combination of naturally occurring substances that are provided to the subject may be provided in amounts or doses (i.e., effective amounts or effective doses) that will elicit a combination of desired effects in the body of the subject.

One or more naturally occurring substances may be provided to a subject to induce thermogenesis, or the production of heat, in the subject's adipocytes, or fat cells. A naturally occurring substance may induce thermogenesis in brown adipose tissue (BAT), or brown fat, of the subject and/or in the subject's white adipose tissue (WAT), or white fat. As a non-limiting example, one or more capsinoids (e.g., dihydrocapsiate, etc.), each in effective amount or an effective dose, may be administered to a subject to induce in the subject's adipocytes.

A subject's metabolism may also be increased by administering one or more naturally occurring substances to the individual. An increase in metabolism includes an increase in the rates at which a subject's body stores and/or consumes energy. The presence of increased levels of cyclic adenosine monophosphate, or cyclic AMP or cAMP, in a subject's blood typically indicates that the subject's metabolism (e.g., the subject's metabolism of fats, sugars, etc.) has increased or improved. The administration of synephrine to a subject is known to result in increased cAMP levels. The skins, or peels, of citrus fruits (e.g., bitter orange (Citrus aurantium) peel extract, etc.) are known sources of synephrine.

A method according to this disclosure may include inhibiting adipogenesis in a subject's adipose cells. Adipogenesis is the creation of fat and/or the storage of energy as fat by adipose cells. An extract of the seeds of African mango, or Irvinia gabonensis, is believed to inhibit adipogenesis.

Naturally occurring substances that reduce a subject's cravings for food, curb the subject's appetite and/or otherwise enable the subject to consume less food may also be administered to a subject in accordance with teachings of this disclosure. In a specific embodiment, administering or otherwise providing synephrine (e.g., in an extract of the peel of a citrus fruit, etc.) to the subject will reduce the subject's cravings for food. Forskolin, which is a component of Coleus forskohlii, or Plectranthus barbatus, is believed to reduce a subject's consumption of food, or to reduce the subject's food intake.

The administration of one or more natural products to a subject may also stimulate the burning of fat by the subject's adipocytes, improve a subject's exercise performance and the effectiveness of exercise by the subject, support the subject's circulatory system and/or otherwise facilitate management of the subject's weight.

Any combination of the foregoing effects may be elicited concurrently with other effects and/or in sequence with other effects. Accordingly appropriate naturally occurring substances may be provided (e.g., administered, etc.) to the subject together (e.g., in a single dose form), separately, or with some naturally occurring substances combined and one or more naturally occurring substances provided individually.

A composition according to this disclosure, which may be referred to as a “weight loss” composition, as a “fat burning” composition or, more simply, as a “composition,” may include any combination of naturally occurring substances that will elicit any of the aforementioned effects in a subject's body. Without limitation, such a composition may include at least one capsinoid, synephrine, African mango and forskolin. The composition may also include at least one capsaicinoid. The at least one capsinoid, the synephrine, the African mango and the forskolin may be the essential ingredients of the composition. In embodiments of the composition that include at least one capsaicinoid, the at least one capsaicinoid may also be an essential ingredient. In a specific embodiment, the composition may include, consist essentially of or even consist of dihydrocapsiate (a capsinoid) or a source thereof (e.g., CH-19 Sweet pepper (Capsicum annuum) fruit extract, etc.); a citrus peel extract (which includes the synephrine), African mango seed extract, Coleus forskohlii root extract (which includes the forskolin) and red pepper (Capsicum annuum) fruit extract (which includes the at least one capsaicinoid).

A weight loss composition according to this disclosure may be used in conjunction with a protein supplement, such as those available from 4Life Research, LC, of Sandy, Utah, under the PRO-TF brand. Use of a weight loss composition according to this disclosure with a protein supplement, such as protein hydrolysates from animal sources (e.g., whey, egg white, etc.), including, but not limited to, hydrolysates with a high degree of hydrolysis (e.g., at least 25% w/w of dipeptides and/or tripeptides, up to about 40% w/w dipeptides and/or tripeptides, etc.), is believed to have synergistic effects on weight management and weight loss. In such a method, the weight loss composition and the protein supplement could be administered or taken together or separately, at appropriate times. As an example, an individual could take a weight loss composition according to this disclosure in the morning, and then take the protein supplement shortly before or shortly after resistance training (e.g., weight lifting, etc.). As another example, an individual could take a weight loss composition according to this disclosure shortly before (e.g., within an hour before, within a half hour before, etc.) exercise or another vigorous physical activity and take a protein supplement shortly after (e.g., within an hour after, within a half hour after, etc.) the exercise other vigorous physical activity.

According to another aspect, this disclosure includes monitoring the thermogenic activity, including regulation of uncoupling protein-1 (UCP1) in adipose tissue (e.g., BAT, etc.). Such a method includes use of thermal imaging techniques to determine a temperature of the adipose tissue and correlating the temperature of the adipose tissue to a certain level of UCP1 activity and/or to regulation of expression of UCP1 in the adipose tissue. Such a technique may be used to determine a pre-treatment UCP1 activity and/or thermogenic activity, as well as any change in UCP1 activity and/or thermogenic activity after the subject has received a weight loss treatment (e.g., a weight loss supplement, such as a weight loss composition according to this disclosure; any other nutritional supplement; any weight loss drug; etc.) over a prolonged period of time (e.g., three (3) days or longer, five (5) days or longer, two (2) weeks or more, etc.). Such information may be useful in identifying the effectiveness with which various compositions promote weight loss.

Other aspects, as well as features and advantages of various aspects, of the disclosed subject matter will become apparent to those of ordinary skill in the art through consideration of the ensuing description and the accompanying claims.

### BRIEF DESCRIPTION OF THE DRAWINGS

FIGS. 1 and 2 are graphs showing the effects of administration of various dosages of an embodiment of a composition according to this disclosure to mice in a first study;

FIG. 3 is a graph showing the percent fat of mice used in a second study, prior to conducting the second study, in which administration of an embodiment of a composition according to this disclosure was evaluated, with and without administration of a protein supplement;

FIGS. 4 and 5 are graphs showing the average consumption of food and water, respectively, by mice of during the second study;

FIGS. 6-11 are graphs showing the change in body weight of the groups of mice in the second study over the course of the second study;

FIG. 12 is a graph showing a plot of rectal temperatures of mice prior to thermal imaging in the second study;

FIG. 13 is an image of mice being subjected to thermal imaging;

FIGS. 14-18 are graphs depicting the temperatures of BAT of the mice, as determined by thermal imaging;

FIG. 19 is a graph showing a plot of rectal temperatures of mice after thermal imaging;

FIG. 20 is an image of a western blot showing amounts of UCP1 in mice at the end of the second study; and

FIG. 21 is a graph showing relative amounts of UCP1 expression by mice at the end of the second study.

### DETAILED DESCRIPTION

A composition according to this disclosure may be formulated to promote weight loss in a subject (e.g., an individual, etc.) to which (or whom) it is administered or by which (or whom) it is taken. In various embodiments, a composition according to this disclosure may include at least one capsinoid, synephrine, African mango and forskolin. In some embodiments, the composition may also include at least one capsaicinoid. The at least one capsinoid may comprise dihydrocapsiate or a source thereof. The synephrine may comprise a component of an extract of a citrus peel, such as a peel extract of bitter orange (Citrus aurantium). The forskolin may be provided in the form of an extract of the root of Coleus forskohlii. The at least one capsaicinoid, if included, may be provided as an extract of the fruit of a red pepper (Capsicum annuum).

Capsinoids, including capsaicin, capsiate, and dihydrocapsiate, are the naturally occurring spicy components of Capsicum annuum peppers. Capsinoids activate thermogenesis via β3-adrenergic receptors and upregulation of uncoupling protein-1 (UCP1), a downstream signal from β3-adrenergic receptors in BAT.

The seed extract of Irvingia gabonensis, also known as African mango, may modulate PPARγ and glycerol-3 phosphate dehydrogenase. In turn, PPARγ and glycerol-3 phosphate dehydrogenase stimulate UCP1 function and expression.

The root extract of the plant Coleus foskolli stimulates intracellular cAMP production, increases UCP1 mRNA and protein in vitro, and reduces weight gain and body fat in vivo.

p-synephrine, the natural stimulant present in Citrus aurantium and other citrus fruits increases energy expenditure in humans, potentially via α-adrenergic and β-adrenergic receptors.

The tables that follow (TABLES 1-4) provide formulas for specific embodiments of compositions according to this disclosure.

TABLE 1

| | Amount per | Amount per 4 |
|---|---|---|
| Ingredient | capsule | capsules serving |
| African Mango (Irvinia | 100 mg (7 mg | 300 mg (21 mg |
| gabonensis) seed extract | active) | active) |
| Citrus fruits extract | 250 mg (12.5 mg | 1000 mg (50 mg |
| (Citrus aurantium, | synephrine) | synephrine) |
| Citrus sinensis, and | | |
| Citrus paradisi) | | |
| [standardized to 5% w/w | | |
| synephrine and 80% w/w | | |
| bioflavinoids] | | |
| Coleus forskohlii root | 166.7 mg (16.7 mg | 500 mg (50 mg |
| extract [10% w/w | forskoli) | forskoli) |
| forskoli] | | |
| Red pepper (Capsicum | 26 mg (0.52 mg | 78 mg (1.56 mg |
| annuum) fruit extract | capsaicinoids; | capsaicinoids; |
| [2% w/w capsaicinoids]; | 0.60 mg | 2.40 mg |
| [2.3% w/w dihydrocapsiate] | dihydrocapsiate) | dihydrocapsiate) |
| CH-19 Sweet pepper | 26 mg (0.60 mg | 78 mg (2.40 mg |
| (Capsicum annuum) fruit | dihydrocapsiate) | dihydrocapsiate) |
| extract [2.3% w/w | | |
| dihydrocapsiate] | | |

TABLE 2

| | Amount per | Amount per 4 |
|---|---|---|
| Ingredient | capsule | capsules serving |
| African Mango (Irvinia | 100 mg (7 mg | 300 mg (21 mg |
| gabonensis) seed extract | active) | active) |
| Bitter orange (Citrus | 250 mg (12.5 mg | 1000 mg (50 mg |
| aurantium) peel extract | synephrine) | |
| [5% w/w synephrine] | | synephrine) |
| Coleus forskohlii root | 166.7 mg (16.7 mg | 500 mg (50 mg |
| extract [10% w/w | forskoli) | forskoli) |
| forskoli] | | |
| Red pepper (Capsicum | 26 mg (0.52 mg | 78 mg (1.56 mg |
| annuum) fruit extract | capsaicinoids; | capsaicinoids; |
| [2% w/w capsaicinoids]; | 0.60 mg | 2.40 mg |
| [2.3% w/w dihydrocapsiate] | dihydrocapsiate) | dihydrocapsiate) |
| CH-19 Sweet pepper | 26 mg (0.60 mg | 78 mg (2.40 mg |
| (Capsicum annuum) fruit | dihydrocapsiate) | dihydrocapsiate) |
| extract [2.3% w/w | | |
| dihydrocapsiate] | | |

TABLE 3

| | Amount per | Amount per 4 |
|---|---|---|
| Ingredient | capsule | capsules serving |
| African Mango (Irvinia | 100 mg (7 mg | 300 mg (21 mg |
| gabonensis) seed extract | active) | active) |
| Bitter orange (Citrus | 250 mg (12.5 mg | 1000 mg (50 mg |
| aurantium) peel extract | synephrine) | synephrine) |
| [5% w/w synephrine] | | |
| Coleus forskohlii root | 166.7 mg (16.7 mg | 500 mg (50 mg |
| extract [10% w/w | forskoli) | forskoli) |
| forskoli] | | |
| Red pepper (Capsicum | 52 mg (1.04 mg | 156 mg (3.12 mg |
| annuum) fruit extract | capsaicinoids; | capsaicinoids; |
| [2% w/w capsaicinoids] | 1.2 mg | 4.8 mg |
| | dihydrocapsiate) | diydrocapsiate) |

TABLE 4

| | | Amount per 4 |
|---|---|---|
| Ingredient | Amount per capsule | capsules serving |
| African Mango (Irvinia | 100 mg (7 mg active) | 300 mg (21 mg |
| gabonensis) seed extract | | active) |
| Bitter orange (Citrus | 250 mg (12.5 mg | 1000 mg (50 mg |
| aurantium) peel extract | synephrine) | synephrine) |
| [5% w/w synephrine] | | |
| Coleus forskohlii root | 166.7 mg (16.7 mg | 500 mg (50 mg |
| extract [10% w/w | forskoli) | forskoli) |
| forskoli] | | |
| CH-19 Sweet pepper | 52 mg (1.2 mg | 156 mg (4.80 mg |
| (Capsicum annuum) fruit | dihydrocapsiate) | dihydrocapsiate) |
| extract [2.3% w/w | | |
| dihydrocapsiate] | | |

The ingredients of a composition according to this disclosure may be combined in a suitable oral dose form. Without limitation, the ingredients of such a composition may be contained by a capsule, such as a gelatin capsule (e.g., a porcine capsule, a bovine capsule, etc.).

While a composition according to this disclosure may be taken or administered at any time, it may be particularly effective when consumed prior to exercise (e.g., an hour before exercising, thirty minutes before exercising, etc.). In addition, or as an alternative, a composition according to this disclosure may be taken or administered in conjunction with consumption of a meal (e.g., within an hour prior to eating, within thirty minutes prior to eating, within thirty minutes after eating, within an hour after eating, etc.). As another option, a composition according to this disclosure may be taken or administered to a subject shortly (e.g., within an hour, within thirty minutes, etc.) after the subject awakens (e.g., in the morning, etc.).

### Example 1

In a first study, an effort was made to determine the toxicity and the acute, five (5) day maximum tolerated dose (MTD) for a product including the proportions of ingredients disclosed in TABLE 1. The effects of food consumption by the subjects and the weights of the subjects were also evaluated.

Three different concentrations of the composition were prepared by mixing different amounts of the composition of TABLE 1 with a vehicle, or carrier, comprising a 0.5% w/w solution of carboxy methyl cellulose (CMC) in deionized water. Three different concentrations of the composition were prepared, with a first concentration including 6.25 mg of the composition per 1.0 mL of the mixture of the composition and the vehicle, a second concentration including 12.5 mg of the composition per 1.0 mL of the mixture and a third concentration including 25.0 mg of the composition per 1.0 mL of the mixture. A control included the vehicle only; i.e., none of the composition.

Mice were used as subjects in the study. More specifically, four (4) week old (wean age) ICR (CD-1) mice from Envigo, Inc., were used as subjects in the study. For eight (8) days prior to stratification and administration of a first dose of the composition, the mice were placed on a special high-fat diet of Rodent Diet with 60% kcal % fat, available from Research Diets, Inc., as Product #D12492. Each mouse remained on this diet until completion of the study.

After the initial eight day period, on the morning of Day 1 of the study, twelve (12) mice were stratified into four (4) groups of three (3) to test the effects of different doses of the composition on the mice. Stratification included weighing each mouse. The mice were stratified on the basis of their weights, with an effort made to keep average weight of the three (3) mice in each group as similar as possible to the average weight of the three (3) mice in each of the other groups. Mice were housed according to their group; that is, three (3) mice per cage. Mice were numbered in each cage, and their ears were notched as follows: Mouse #1—left ear, Mouse #2—right ear, Mouse #3—no notch.

At the end of the day (i.e., in the evening) of each of Day 1 through Day 5 of the study, each mouse received a dose, by oral gavage with a large gauge feeding needle, of one of the above-described mixtures (i.e., concentrations of the composition) or of the vehicle. The dose amounts used in the study were 20 mL of the mixture or control for each kilogram of the subject's body weight. For a mouse weighing 0.025 kg, about 0.5 mL of one of the three mixtures or the control was administered each day. Each of the three (3) mice in a first group received the 6.25 mg/mL solution at the at the 20 mL/kg dose rate, for a dosage of 125 mg of the composition per 1 kg of body weight each day (i.e., a daily dose of 125 mg/kg). Each of the three (3) mice in a second group received the 12.5 mg/mL solution at the 20 mL/kg dose rate for a daily dose of 250 mg/kg. Each of the three (3) mice in a third group received the 25 mg/mL solution at the 20 mL/kg dose rate, for a daily dose of 500 mg/kg. A fourth group served as a control group, in which each of the three (3) mice received the vehicle, or carrier, only at the 20 mL/kg dose rate. The mixtures and the control were mixed thoroughly prior to each dosing, as each mixture could separate, or become heterogeneous, over short periods of time.

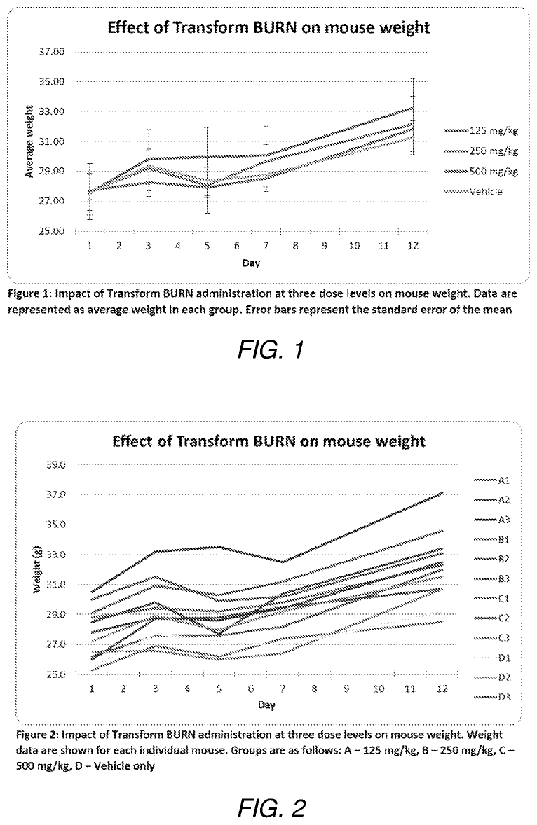

Each mouse was weighed again on the evening of each of Day 3, Day 5, Day 7 and Day 12, and the weights were recorded. FIG. 1 shows the average weight of the mice in each group over the course of the study. The error bars in FIG. 1 represent the standard error of the mean (i.e., the standard deviation from the mean). FIG. 2 shows the weight of each mouse over the course of the study. Mice A1, A2 and A3 received a daily dose of 125 mg/kg on each of Day 1 through Day 5; mice B1, B2 and B3 received a daily dose of 250 mg/kg on each of Day 1 through Day 5; mice C1, C2 and C3 received a daily dose of 500 mg/kg on each of Day 1 through Day 5; and mice D1, D2 and D3, the control mice, only received the vehicle on each of Day 1 through Day 5.

On the evening of each day of the study, the technician also observed and made a record of the morbidity for each mouse. None of the mice died during the course of the study. At the same time, the technician recorded any incidental observations.

Food consumption over the course of the study was determined by weighing the food provided to each group of mice at the outset of the study (i.e., on Day 1) and recording that value, weighing any additional food provided to each group of mice during the study and recording that value, and then weighing the food for that group of mice remaining at the end of the study (i.e., after Day 12) and recording that value. The weight of the food that was initially provided to each group of mice was added to the additional food provided to that group of mice during the course of the study to determine the total weight of food provided to the group of mice during the course of the study. The weight of the food remaining for that group of mice after the end of the study was then subtracted from the total weight of food provided to determine the amount of food consumed by that group of mice over the course of the study. The amount of food provided to and consumed (“eaten”) by each group of mice during the course of the study is set forth in the table that follows.

TABLE 5
Food Provided and Consumed by Dose Group

| | | Additions | | Remaining | |
|---|---|---|---|---|---|
| Group | Day 1 | Day 2 | Day 7 | Day 12 | Eaten |
| A - 125 mg/kg | 45.8 | 30.7 | 45.5 | −29 | 93 |
| B - 250 mg/kg | 43 | 31.2 | 37.4 | −22.9 | 88.7 |
| C - 500 mg/kg | 43.1 | 31.6 | 49.3 | −41.2 | 82.8 |
| D - Vehicle | 43.6 | 29.8 | 46.8 | −37.2 | 83 |

Food consumption appeared to track with average weights; the group (Group A, which received a daily dose of given 125 mg/kg of the composition) that consumed the most food also had the highest final average weight (see FIGS. 1 and 2).

From this study, it appears that administration of a composition with ingredients in the proportions set forth in TABLE 1, when compared with the control group (Group D), did not appear to have any adverse effect on weight gain in any of the mice at any dose level (i.e., Groups A, B and C). For all three (3) groups that received (i.e., Groups A, B and C) some of the composition, the rate at which the mice gained weight was substantially steady or decreased within a couple of days after the composition was administered and for a couple of days following administration of the final dose of the composition. The weight of each mouse increased between Day 7 and Day 12 (i.e., the end of the study). A similar decrease in the rate at which the mice lost weight, and then a similar increase in the rate at which mice gained weight was observed in the control group (Group D).

No adverse reactions were noted in any of the treatment groups. At the end of the study, all twelve (12) of the mice appeared to be normal and healthy. One mouse in the control group (Group D) had irritation in its right eye, which persisted throughout the study, but that mouse's eye irritation did not appear to affect the health of that mouse, and was not deemed as grounds for removing that mouse from the study. Every dose of the composition and the vehicle that was administered on Days 1-5 of the study was well tolerated.

Prior to the start of the study, there was some concern that the formulation or the test agent could cause gastrointestinal toxicity. However, observations during the course of the study did not reveal any evidence of diarrhea or abnormal stools (aside from the green/blue stool color that was typical once the mice began consuming the high-fat diet, before the composition and vehicle were administered). Further, there was no apparent reduction in food consumption over the course of the study.

From the observations made during the study, the composition, at least in the daily dosage rates that were tested, does not appear to be toxic. Further, it appears that the composition can be administered at any of the tested daily dosage rates (i.e., 125 mg/kg, 250 mg/kg, 500 mg/kg) and at daily dosage rates that exceed 500 mg/kg without any adverse effects on the subject (e.g., increased mortality, increased morbidity, intestinal complications, decreased food consumption, etc.).

### Example 2

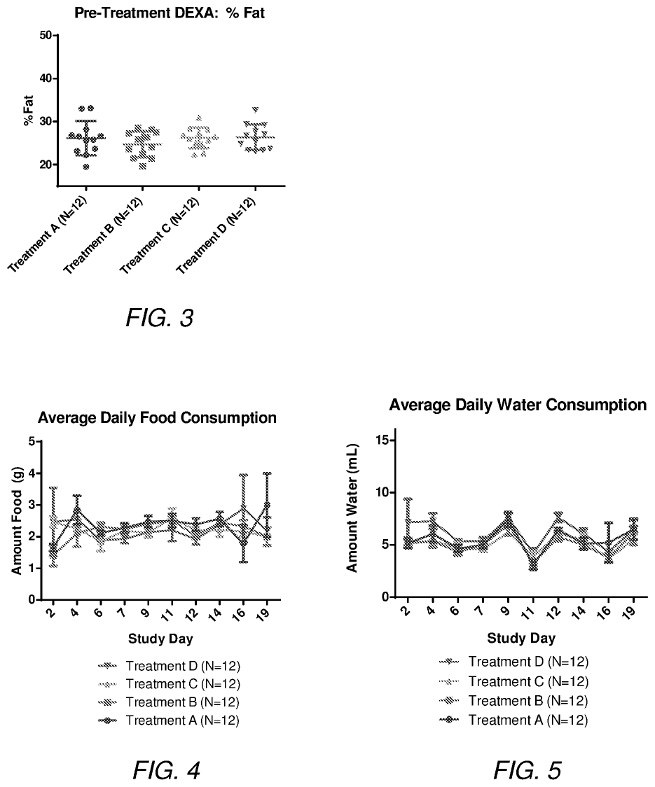

In another study, several newly weaned, four (4) week-old C57BL/6J mice from The Jackson Laboratory of Bay Harbor, Maine, were ear-notched for identification and housed individually in positively ventilated, high efficiency particulate air (HEPA)-filtered polysulfonate cages. The room in which the mice were kept was lighted entirely with artificial fluorescent lighting, with controlled 12 hour light and dark cycles (light from 6:00 a.m. to 6:00 p.m.; dark from 6:00 p.m. to 6:00 a.m.). The normal temperature and relative humidity in the room were 22±4° C. and 50±15%, respectively. FIG. 3 shows the percent fat, by weight, of each mouse at four (4) weeks old.

The mice were provided with high-fat diets. Specifically, each mouse was fed the rodent diet with 60% kcal % fat available as OpenSource Diets® D12492 from Research Diets, Inc., of New Brunswick, New Jersey. The food and water were provided ad libitum. After four (4) weeks on the rodent diet with 60% kcal % fat, these diet-induced obese (DIO) mice, then eight (8) weeks old, were randomly stratified into three (3) groups, with ten (10) or eleven (11) mice in each group (n=10-11). A first group (Group A in the figures) of mice served as a control group, and received the above-mentioned vehicle in a dosage amount of 20 mL of the 0.5% w/w CMC vehicle, without composition, per kilogram (kg) of body weight each day. The vehicle was administered in the morning.

A second group (Group B in the figures) of mice received a dosage amount of 250 mg/kg body weight of the composition of TABLE 1 (approximately equivalent to a human dose of four (4) capsules per day) dissolved in 20 mL of the 0.5% CMC vehicle each day of the study. The composition was administered in the morning.

A third group (Group C in the figures) of mice received a daily dose of 500 mg/kg body weight of the composition each day, dissolved in 20 mL of the of the 0.5% w/w CMC vehicle. The third group was added to determine whether or not a small increase in dosage would have any significant effect on the ability of the composition to control weight gain in mice. The composition was administered in the morning.

A fourth group (Group D in the figures) received a daily dose of 250 mg/kg body weight of the composition each day, as well as protein supplementation. More specifically, each mouse in Group D received a human equivalent daily dose, based on the weight of that mouse, of a composition including hydrolyzed protein obtained from animal sources. Even more specifically, each mouse in Group D received a human equivalent daily dose of 10 g (about 2 g/kg body weight) of the 4LifeTransform® PRO-TF® protein supplement available from 4Life Research, LC, of Sandy, Utah, which includes whey protein concentrate, extensively hydrolyzed proteins from whey and egg whites, and extracts of bovine colostrum and egg yolk. The human equivalent daily dose of the protein supplement for each mouse was dissolved in 10 mL of deionized water. The composition was administered in the morning. The protein supplement was administered in the afternoon.

The vehicle or composition dissolved in vehicle was administered to each mouse by oral gavage each day during the ninth through twelfth weeks of each mouse's life. In the fourth group (Group D), which received the protein supplement, the protein supplement was administered about four (4) hours after administration of the composition according to this disclosure, also by oral gavage.

Food consumption was measured as described in EXAMPLE 1, but on a more frequent basis—three times per week. The average (per mouse) food consumption data for each group is illustrated by the graph of FIG. 4. Water consumption was measured in the same manner, and is shown in the graph of FIG. 5.

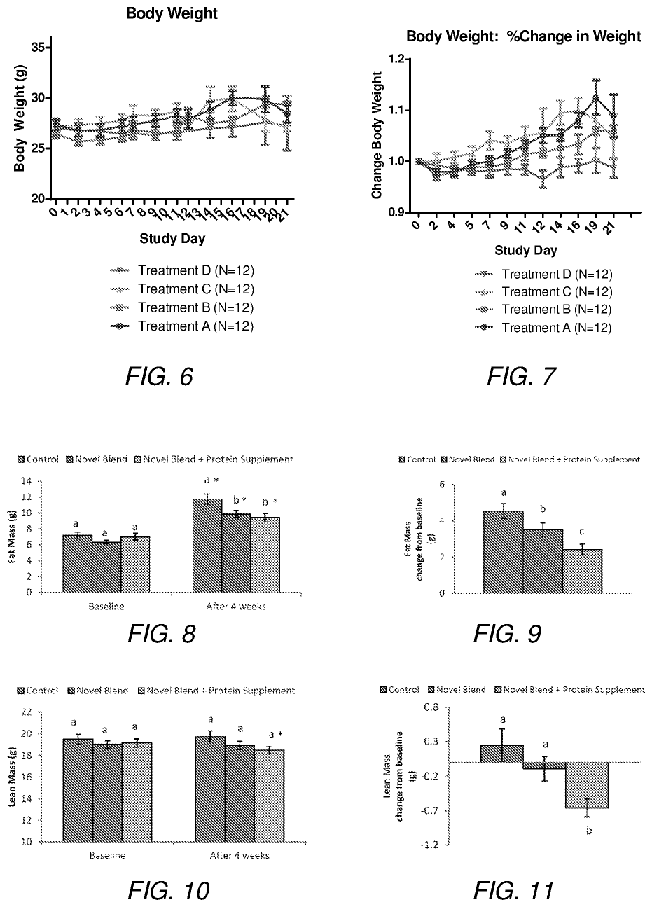

Each mouse was weighed three times each week, just before receiving the vehicle (Group A) or the composition dissolved in the vehicle (Groups B and D). Each mouse was first anesthetized with isoflurane (2-chloro-2-(difluoromethoxy)-1,1,1-trifluoro-ethane), then weighed by way of dual-energy x-ray absorptiometry (DEXA). The graph of FIG. 6 shows the average body weight for the mice of each group over the course of the study. The graph of FIG. 7 shows the average percent change in body weight for each group over the course of the study (i.e., the variation in body weight since Day 0, at the outset of the study).

In addition, while each mouse was anesthetized, bone density, fat mass, and lean muscle mass measurements were obtained by way of DEXA, as depicted by FIG. 7, which indicates that the percentage of fat in the mice, pretreatment, is not significantly different amongst the mice used in the study. FIG. 8 shows the average (per mouse) fat mass of each group at Day 0 and at Day 28. FIG. 9 shows the average change in fat mass for each group from Day 0 to Day 28. FIG. 10 shows the average lean mass of each group at Day 0 and at Day 28. FIG. 11 shows the average change in lean mass for each group from Day 0 to Day 28. As illustrated by FIG. 9 the composition that was administered to the mice of Groups B and D promoted weight loss. In addition, FIG. 9 shows that when the composition that was administered to the mice of Group B is used in conjunction with protein supplementation, as occurred with the mice of Group D, even further weight loss can be achieved, indicating that a composition according to this disclosure may function synergistically with protein supplementation.

Pre-treatment body composition and metabolic data were collected immediately before each mouse received its first dose of the vehicle (Group A) or of a mixture including the composition and the vehicle (Groups B and D).

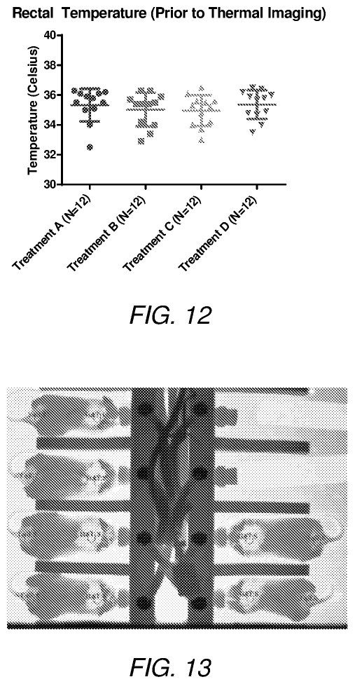

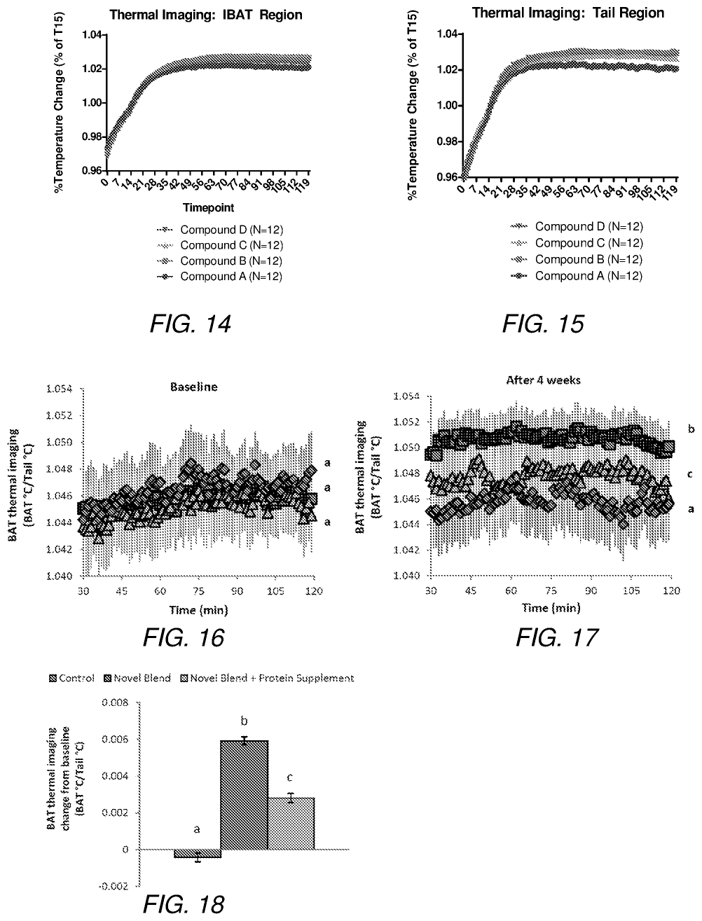

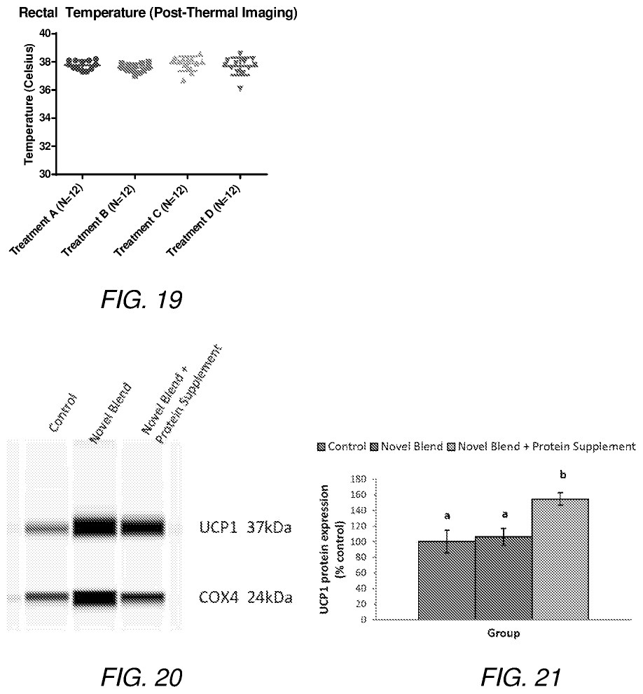

Prior to gathering body composition and metabolic data, each mouse was anesthetized with isoflurane. The rectal temperature of each mouse was then obtained (FIG. 12). Fur was removed from the subscapular region and at the base of the tail of each mouse. After the fur was removed, each mouse was placed on an imaging platform that had been heated to 37° C. to reach and maintain a constant body temperature. The temperature of the brown adipose tissue of each mouse was then obtained by surface thermal imaging, which employs infrared radiation, using the FLIR A6703sc thermal camera and researchIR™ software available from FLIR Systems of Wilsonville, Oregon. FIG. 13 is an image obtained by such thermal imaging. As shown in FIGS. 14 and 15, the baseline temperature in the thermal imaging analyses is not reached until fifteen (15) minutes, meaning that it takes about fifteen (15) minutes for the bodies of the mice to warm to the temperature of the imaging platform. In this study, the intrascapular brown adipose tissue (IBAT), which is located between the mouse's shoulders (FIG. 14), and the brown adipose temperature of the mouse's tail region (FIG. 15) were thermally imaged. The thermal imaging analysis provides information (e.g., tissue temperatures, etc.) that corresponds to the activity of uncoupling protein-1 (UCP1) mediated thermogenesis in the tissues that are imaged. FIG. 16 shows the BAT temperature of each mouse on Day 0, at the outset of the study. FIG. 17 shows the BAT temperature of each mouse on Day 28. FIG. 18 is a graph that shows the average (per mouse) change in BAT temperature that occurred in each group from Day 0 to Day 28. After thermal imaging, the rectal temperature of each mouse was again obtained (FIG. 19). The data show that the rectal temperatures of the mice increased during thermal imaging, which was expected as the temperature of the thermal imaging platform exceeded the temperature of the environment in which the mice are kept.

Blood samples were also obtained while each mouse was anesthetized. More specifically, samples of about 200 μL of whole blood were collected from the mice by retro-orbital eye bleed into BD™ P800 vacutainers available from Becton, Dickinson and Company of Franklin Lakes, New Jersey. The blood samples were then processed in a refrigerated centrifuge set to a temperature of 4° C. and spun at 14,000 rpm for ten (10) minutes. The plasma was then analyzed to assess levels of insulin, leptin, and adiponectin using the Mouse Metabolic Kit (K15124C-3) and the Mouse Adiponectin Kit (K152BXC-1) available from Meso Scale Diagnostics LLC of Rockville, Maryland. Leptin is a hormone made by adipose cells that helps to regulate energy balance by inhibiting hunger. Increased amounts of leptin correspond to an increase in satiety, or feeling full. Adiponectin is a protein that is involved in regulating glucose levels and fatty acid breakdown. Increased levels of adiponectin correspond to increased fat metabolism, or burning.

Indirect calorimetry measurements were also obtained. Indirect calorimetry was performed using a comprehensive cage monitoring system (CCMS), available from Columbus Instruments International Corporation of Columbus, Ohio, as the Oxymax™ Lab Animals Monitoring System. Each mouse was placed, by itself, in a CCMS for a period of seventy-two (72) hours. Food and water were provided ad libitum during that period. In addition, oxygen consumption, carbon dioxide production, and heat production were measured every 30-60 minutes throughout the course of each seventy-two (72) hour period.

Body composition and metabolic data were also collected at the end of the study, immediately after each mouse received its final dose of a mixture including the composition or the vehicle. Again, after each mouse was anesthetized, a rectal temperature measurement was obtained, the mouse was subjected to thermal imaging, and then another rectal temperature measurement was obtained. Bone density measurements were also obtained. Thereafter, indirect calorimetry was performed.

At the conclusion of the study, IBAT was harvested from each mouse, at about 2½ hours after each mouse received its final dose of the vehicle or composition. The IBAT samples were snap-frozen on dry ice. Mitochondria were isolated from the IBAT using the mitochondrial isolation kit available from Abcam Company of Cambridge, United Kingdom, as product no. ab110168. The protein in each IBAT sample was quantified using a DC™ Protein Assay available from Bio-Rad Laboratories, Inc., of Hercules, California. The UCP1 protein in each sample was quantified using the Wes™ system available from proteinsimple of San Jose, California, using 0.5 μg protein loading, Ucp1 antibody at a 1:100 dilution, and Cox4 antibody (mitochondrial house-keeping) at a 1:100 dilution. The results of this protein assay are depicted by the image of FIG. 20 and the graph of FIG. 21.

Four (4) of the mice died during the study. Two (2) of the deaths were attributed to errors in the manner in which the composition was administration, not to the composition itself. Data obtained from observing the dead mice will be omitted from the data in the study.

Notably, as shown in FIGS. 4 and 5, no significant differences in food consumption (FIG. 4) or water consumption (FIG. 5) were observed between groups.

From the data obtained during the study, statistical analyses were conducted using Statistica® software available from StatSoft Inc. of Tulsa, Oklahoma. The baseline and post-treatment outcome measures were analyzed using a one-way ANOVA followed by a Newman-Keuls post-hoc test for determination of significance among groups. Differences among groups were considered significant if the probability of type I error was <5% (p<0.05). Baseline measures were not significantly different among groups.

Over the course of the study, as shown in FIG. 6, all of Groups A, B, and D gained weight (p<0.05). As shown by FIGS. 6-9, at the end of the study, i.e., after 4 weeks of daily oral supplementation, mice on high-fat diet treated with a composition according to this disclosure (i.e., the mice of Group B) gained significantly less weight than mice on high-fat diet treated with vehicle control (i.e., the mice of Group A) (F2,29=6.89; p=0.0168 vs. control). The composition alone began exhibiting desired effects on body weight gain (e.g., less weight gain than the control group, weight loss) after 3 weeks of daily supplementation (F2,29=10.85; p=0.0256 vs. control, p=0.0265 vs. composition+protein). The addition of protein supplementation (i.e., in the mice of Group D) led to a further reduction in body weight gain (p=0.0028 vs. control), as illustrated by FIGS. 6-9. When the composition was administered in conjunction with protein supplementation, desired effects on body weight gain were observed in about 2 weeks or less (F2,29=7.03; p=0.0022 vs. control, p=0.0508 vs. composition alone). Since the mice of Group D consumed as much food as the mice of the other groups, it appears that the reduction in body weight gain was not due to reduced consumption of food, but to the effects of the composition and the protein supplement.

Mice that received the composition of this disclosure alone (Group B) and in combination with protein supplementation (Group D) had significantly less fat mass than animals that received vehicle (F2,29=5.29; control vs. novel blend p=0.0174 and vs. composition+protein p=0.0115) without a significant effect on lean muscle mass, as illustrated by FIGS. 10 and 11.

As shown in the graphs of FIGS. 14, 15, 17, and 18, the composition of this disclosure alone and in combination with protein supplementation led to significantly greater BAT temperature than the BAT temperature of mice of the control group (Group A) (F2, 3837=111.28; control vs. composition p<0.0001 and vs. composition+protein p<0.0001). Additionally, data revealed that the composition alone increased BAT temperature to a greater extent than the composition+protein supplement (p<0.0001). UCP1 expression in BAT was also significantly greater in animals that received the composition of this disclosure, both alone and in combination with protein supplementation (F2,24=4.90; p=0.0195 vs. control, p=0.0160 vs. composition).

No treatment effects were observed on oxygen consumption, heat production, and respiratory exchange ratio.

The data presented in TABLE 5 reveal that the novel blend alone or in combination with the protein supplement did not significantly affect blood levels of insulin, leptin, and adiponectin (F2,29=0.64, F2,29=2.41, and F2,29=0.28 respectively). However, there was a trend of lower leptin levels in animals treated with the composition of this disclosure when used in conjunction with protein supplementation (p=0.089 vs. control).

TABLE 6
Blood Parameters (Mean Values + S.E.M.)

| | | | Adiponectin |
|---|---|---|---|
| | Insulin (pg/ml) | Leptin (pg/ml) | (pg/ml) |
| Group A | 2415 + 1119 | 16763 + 7093 | 24.8 + 5.9 |
| Group B | 2994 + 1223 | 14070 + 7621 | 24.4 + 5.0 |
| Group D | 2338 + 1864 | 10275 + 5610 | 23.4 + 2.4 |

The data obtained from the study indicate that administration of a composition according to this disclosure attenuates gains in body weight and fat mass within about three (3) weeks, even when used by subjects who eat high-fat diets. Such a composition may also reduce fat mass and body weight in a subject to whom the composition is administered. The data also indicate that these positive effects on fat mass and body weight were improved even further when a composition according to this disclosure is administered in conjunction with protein supplementation.

In addition, the data from the thermal imaging performed in the study indicates that a composition according to this disclosure, when administered alone or with a protein supplement, increases the temperature of brown adipose tissue in a subject. An increase in the temperature of brown adipose tissue is, in turn, indicative of an increase in thermogenesis in the brown adipose tissue. The brown adipose tissue of subjects who received the composition and a protein supplement with hydrolyzed whey protein also exhibited elevated levels of the thermogenic biomarker UCP1. Although the dose of composition used in the study did not upregulate UCP1 in brown adipose tissue, it is believed that a higher dose of a composition according to this disclosure will upregulate UCP1 in brown adipose tissue. An increase in the activity of UCP1 in brown adipose tissue may be accompanied by increases in the activity of UCP1 in other types of tissues.

The thermogenic effect of a composition of this disclosure, when administered with a protein supplement (e.g., a protein supplement that includes hydrolyzed whey, etc.), may contribute to a greater attenuation of increases in body weight and/or fat mass than administration of the composition alone.

Compositions according to this disclosure do not appear to have any significant effects on the lean muscle mass, metabolism (i.e., energy expenditure), or levels of insulin, adiponectin, or leptin in the blood of subjects to whom they are administered.

Although the foregoing disclosure sets forth many specifics, these should not be construed as limiting the scope of any of the claims, but merely as providing illustrations of some embodiments and variations of elements and/or features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

What is claimed:

1. A method for burning fat in a body of a subject, comprising:
inducing thermogenesis in adipocytes of a the subject by administering at least one capsinoid to the subject in an amount effective for inducing thermogenesis in brown adipocytes of the subject;
increasing an amount of cyclic adenosine monophosphate (cAMP) released by the brown adipocytes of the subject by administering synephrine to the subject in an amount effective for increasing cAMP;
inhibiting adipogenesis in adipocytes of the subject by administering African mango to the subject in an amount effective for inhibiting adipogenesis in the brown adipocytes of the subject;
elevating uncoupling protein 1 (UCP1) levels in the brown adipocytes of the subject by administering the at least one capsinoid, the African mango, and forskolin to the subject in amounts effective for elevating the UCP1 levels;
reducing hunger cravings by the subject by administering the forskolin to the subject in an amount effective for reducing food intake; and
administering at least one capsaicinoid to the subject.

2. The method of claim 1, wherein administering the at least one capsinoid comprises administering dihydrocapsiate to the subject.

3. The method of claim 1, wherein administering the synephrine comprises administering a citrus peel extract to the subject.

4. The method of claim 1, wherein administering the at least one capsaicinoid comprises administering red pepper fruit extract to the subject.

5. The method of claim 1, wherein administering the forskolin comprises administering a Coleus forskohlii root extract to the subject.

6. The method of claim 1, wherein:
administering the at least one capsinoid comprises administering about 5 mg of the at least one capsaicinoid to the subject;
administering the synephrine comprises administering about 50 mg of the synephrine to the subject;
administering the at least one capsaicinoid comprises the about 2 mg of the at least one capsaicinoid to the subject;
administering the African mango seed extract comprises administering about 300 mg of African mango seed extract to the subject; and
administering the forskolin comprises administering about 50 mg of the forskolin to the subject.

7. The method of claim 1, wherein administering the synephrine comprises administering the synephrine in an amount effective for reducing hunger cravings.

8. A method for burning fat in a body of a subject, comprising:
inducing thermogenesis in brown adipocytes of an subject;
increasing an amount of cyclic adenosine monophosphate (cAMP) released by the brown adipocytes of the subject;
inhibiting adipogenesis in the brown adipocytes of the subject; and
reducing hunger cravings by the subject,
the acts of inducing thermogenesis in the brown adipocytes, increasing the amount of cAMP released by the brown adipocytes, inhibiting adipogenesis in the brown adipocytes, and reducing hunger cravings comprise:
administering to the subject:
a composition tailored to upregulate expression of uncoupling protein 1 (UCP1) in adipose tissue of a subject to facilitate fat metabolism; and
a protein supplement.

9. The method of claim 8, wherein administering to the subject the composition tailored to upregulate express of UCP1 comprises administering at least one capsinoid to the subject.

10. The method of claim 8, wherein administering to the subject the composition the protein supplement comprises administering hydrolyzed protein from at least one animal source to the subject.

11. The method of claim 10, wherein administering the hydrolyzed protein from at least one animal source includes administering at least 25% w/w of dipeptides and/or tripeptides to the subject.

12. The method of claim 8, wherein:
administering to the subject the composition tailored to upregulate expression of UCP1 comprises administering the composition tailored to upregulate expression of UCP1 prior to physical activity; and
administering to the subject the protein supplement comprises administering the protein supplement to the subject following physical activity.

13. A method for burning fat in a body of a subject, comprising:
inducing thermogenesis in brown adipocytes of the subject;
increasing an amount of cyclic adenosine monophosphate (cAMP) released by the brown adipocytes of the subject;
inhibiting adipogenesis in the brown adipocytes of the subject;
elevating uncoupling protein 1 (UCP1) levels in the brown adipocytes of the subject; and
reducing hunger cravings by the subject,
the acts of inducing thermogenesis in the brown adipocytes, increasing the amount of cAMP released by the brown adipocytes of the subject, inhibiting adipogenesis in the brown adipocytes, and reducing hunger cravings comprise:
administering to the subject a composition consisting of:
dihydrocapsiate;
African mango seed extract;
citrus peel extract;
Coleus forskohlii root extract; and
red pepper fruit extract
in proportions and in an amount effective for inducing thermogenesis in the adipocytes, increasing the amount of cAMP) released by the cells, inhibiting adipogenesis in the adipocytes, and reducing hunger cravings.

14. The method of claim 13, wherein administering comprises administering the composition with the dihydrocapsiate being Sweet pepper fruit extract with 2.3% dihydrocapsiate, by weight.

15. The method of claim 13, wherein administering comprises administering the composition with the red pepper fruit extract being 2% capsaicinoids, by weight.

16. The method of claim 13, wherein administering comprises administering the composition with the dihydrocapsate and/or the red pepper fruit extract being included in an amount that induces thermogenesis.

17. The method of claim 13, wherein administering comprises administering the composition with the African mango being included in an amount that deceases total cholesterol and LDL cholesterol levels.

18. The method of claim 13, wherein administering comprises administering the composition with the African mango being included in an amount that inhibits adipogenesis in brown adipocytes.

19. The method of claim 13, wherein administering comprises administering the composition with the citrus peel extract comprising 5% synephrine, by weight, and 80% flavonoids, by weight.

20. The method of claim 13, wherein administering comprises administering the composition with the citrus peel extract being included in an amount that reduces hunger cravings.

21. The method of claim 13, wherein administering comprises administering the composition with the citrus peel extract being included in an amount that increases cAMP release by the brown adipocytes of the subject.

22. The method of claim 13, wherein administering comprises administering the composition with the Coleus forskohlii root extract being included in an amount that reduces food intake.
